(19) United States
(12) Patent Application Publication
(10) Pub. No.: US 2015/0018718 A1
(43) Pub. Date: Jan. 15, 2015

(54) Sensor for Measuring the Tilt of a Patient's Pelvic Axis

(71) Applicant: ARTHROMEDA, INC., Ayer, MA (US)

(72) Inventor: Mehran S. Aghazadeh, Newton, MA (US)

(21) Appl. No.: 14/330,261

(22) Filed: Jul. 14, 2014

Related U.S. Application Data
(60) Provisional application No. 61/845,567, filed on Jul. 12, 2013.

(51) Int. Cl. A61B 5/107; G01B 21/22; A61B 5/00

(52) U.S. Cl. CPC A61B 5/1071; A61B 5/4571; G01B 21/22
USPC 600/587

(57) ABSTRACT

A sensor and method for providing orientation data regarding a patient's pelvic axis is disclosed. The sensor includes a three axis tilt sensor; a wireless communication module; a power source; and a microcontroller. The microcontroller is in electronic communication with the three axis tilt sensor, the wireless communication module, and the power source so as to control the three axis tilt sensor and the wireless communication module so that the sensor is configured to measure and report on an orientation of a pelvic axis of a patient to whom the sensor has been connected in a known orientation. The microcontroller is able to control the sensor in order to maximize the power draw and extend the life of the portable power source.

### CROSS-REFERENCE TO RELATED APPLICATIONS

This application claims benefit under 35 U.S.C. §119(e) of U.S. Provisional Patent Application Ser. No. 61/845,567, filed Jul. 12, 2013.

### BACKGROUND OF THE INVENTION

Successful hip prosthetic surgery requires precise intra-operative placement and positioning of replacement structures as implants within the patient such that the in vivo function of the reconstructed joint is optimized biomechanically and biologically. For the surgeon, it is necessary to ensure that the replacement structural components are implanted correctly and function in situ properly in order to avoid intraoperative and post-operative complications, as well as to ensure a long-lasting action and use for the implanted prosthesis.

There are three critical parameters for achieving a successful hip arthroplasty procedure: (1) position angles of the cup; (2) position angle of the stem; and (3) longitudinal placement of the stem.

A malpositioned hip prosthesis will not adequately restore the joint's biomechanics, will not function properly, and is at increased risk of intra-operative and post-operative complications. Such complications can include, without limitation, dislocation, impingement, fracture, implant failure, aseptic loosening, and subsidence. A malpositioned prosthetic implant is particularly susceptible to dislocation and early loosening because the prosthesis will not be well fitted or supported within the host's native bone.

The biggest problem routinely faced by surgeons today concerning human hip replacement procedures is how to achieve proper acetabular prosthetic implant alignment. It is generally agreed among orthopedic surgeons that the ideal anatomic position (for most patients) for positioning the acetabular prosthetic implant within the native bone of the host's hip is at 45° of inclination.

A second important angle is the angle of forward flexion, which ideally is at 20° of forward flexion. More recent advanced techniques emphasize “combined anteversion” of the reconstructed hip, rather than the cup's absolute angle of forward flexion. Combined anteversion is the sum of the angle of forward flexion of the cup plus the angle of anteversion of the stem. Since there is limited space for changing the stem's angle of anteversion, adjusting the position of the cup to that of the stem is critical to improving stability of the reconstructed hip and reducing impingement.

However, precise measurement of these specific angles, and therefore proper placement of the prostheses, has been difficult to achieve, mostly because two of these angles are relative to the patient's pelvis and the patient is covered by sterile surgical drapes during the course of the hip replacement operation.

### BRIEF SUMMARY OF THE INVENTION

An aspect of the invention is a sensor for providing orientation data regarding a patient's pelvic axis. The sensor includes a three axis tilt sensor; a wireless communication module; a power source; and a microcontroller in electronic communication with the three axis tilt sensor, the wireless communication module, and the power source. The microcontroller controls the three axis tilt sensor and the wireless communication module such that the sensor is configured to measure and report on an orientation of a pelvic axis of a patient to whom the sensor has been connected in a known orientation.

In one embodiment, the microcontroller further controls the three axis tilt sensor and the wireless communication module so that the battery can power the three axis tilt sensor, the wireless communication module, and the microcontroller for a period of at least one hour.

In one embodiment, the microcontroller further controls the three axis tilt sensor and the wireless communication module so that the battery can power the three axis tilt sensor, the wireless communication module, and the microcontroller for a period of at least four hours.

In one embodiment, the three axis tilt sensor includes a microelectromechanical accelerometer and a magnetometer.

In one embodiment, the sensor further includes a computer processor in wireless communication with the sensor, the computer processor including application software and a graphical user interface for converting data from the sensor into orientation information for the patient's pelvic axis and displaying the orientation information to a user.

An aspect of the invention is a method for sensing and reporting on the orientation of a patient's pelvic axis. The method includes the step of providing a sensor including a three axis tilt sensor; a wireless communication module; a power source; and a microcontroller in electronic communication with the three axis tilt sensor, the wireless communication module, and the power source. The method further includes the steps of mounting the sensor on the patient in a known orientation with respect to the patient's pelvic axis; sensing orientation data regarding the pelvic axis using the three axis tilt sensor; and communicating the orientation data using the wireless communication module.

In one embodiment, the method includes the microcontroller controlling the three axis tilt sensor and the wireless communication module so that the battery can power the three axis tilt sensor, the wireless communication module, and the microcontroller for a period of at least one hour.

In one embodiment, the method includes the microcontroller controlling the three axis tilt sensor and the wireless communication module so that the battery can power the three axis tilt sensor, the wireless communication module, and the microcontroller for a period of at least four hours.

In one embodiment, the method includes the three axis tilt sensor including a microelectromechanical accelerometer and a magnetometer.

In one embodiment, the method includes receiving of the wireless transmission of orientation data by a computer processor having an application program and a graphical user interface.

In one embodiment, the method includes processing the orientation data by the application program and displaying orientation information regarding the patient's pelvic axis using the graphical user interface.

### BRIEF DESCRIPTION OF THE DRAWINGS

The present invention can be more easily understood and better appreciated when taken in conjunction with the accompanying drawings, in which:

FIG. 1 depicts various anatomical planes and axes of the human body;

FIG. 2 depicts the angles of inclination (A), forward flexion (B), and anteversion (C);

FIG. 3 depicts a transverse section through a distal femur at the level of the lateral (L) and medial (M) condyles

FIG. 4 illustrates a layout for a sensor of the invention; and

FIG. 5 illustrates a package diagram for a sensor of the invention.

### DETAILED DESCRIPTION OF THE INVENTION

The invention is directed generally to a sensor that can be placed on a patient in a known angular relationship with the pelvic axis in order to provide measurements that allow a surgeon to implant an acetabular cup at a desired angle, including when there is movement of the patient during surgery. This sensor can also be placed on an instrument, which is routinely used for handling and placing the acetabular implant, in order to measure the implant's absolute position angles while being or when placed in patient's bony pelvis.

The invention can be used with the systems and methods of WO2013/049534, which is hereby incorporated in its entirety as if its full contents were repeated here. In addition, the sensor can be placed using the systems and methods described in U.S. provisional patent application 61/845,523, filed on Jul. 12, 2013 and entitled “Systems and Methods for Aligning a Medical Device with a Pelvic Axis,” which is hereby incorporated in its entirety as if its full contents were repeated here. Those systems and methods provide an intra-operative surgical positioning assessment and angle determination made by anatomic alignment.

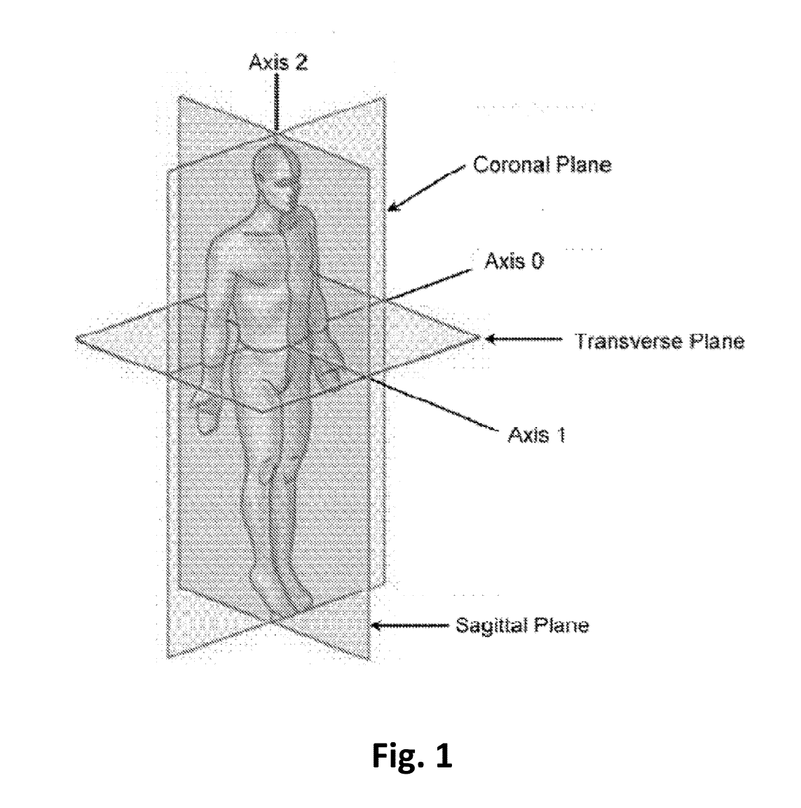

The method and system determine the patient's true pelvic position/tilt by using the geometric planes as anatomical reference planes, i.e., alignment and angles are measured relative to the true features of the patient, not just to, for example, the operating table. As shown in FIG. 1, the transverse plane divides the human body into top and bottom sections; the coronal plane divides the body into front (anterior) and back (posterior) portions; and the sagittal plane divides the body into left-sided and right-sided portions.

Also by definition and anatomic convention, “Axis 0” is the common line between the transverse and coronal planes; “Axis 1” is the common line between the transverse and sagittal planes; and “Axis 2” is the common line between the coronal and sagittal planes. The pelvic axis is any line defined by the pelvis and generally parallel to Axis 0 or generally perpendicular to the sagittal plane.

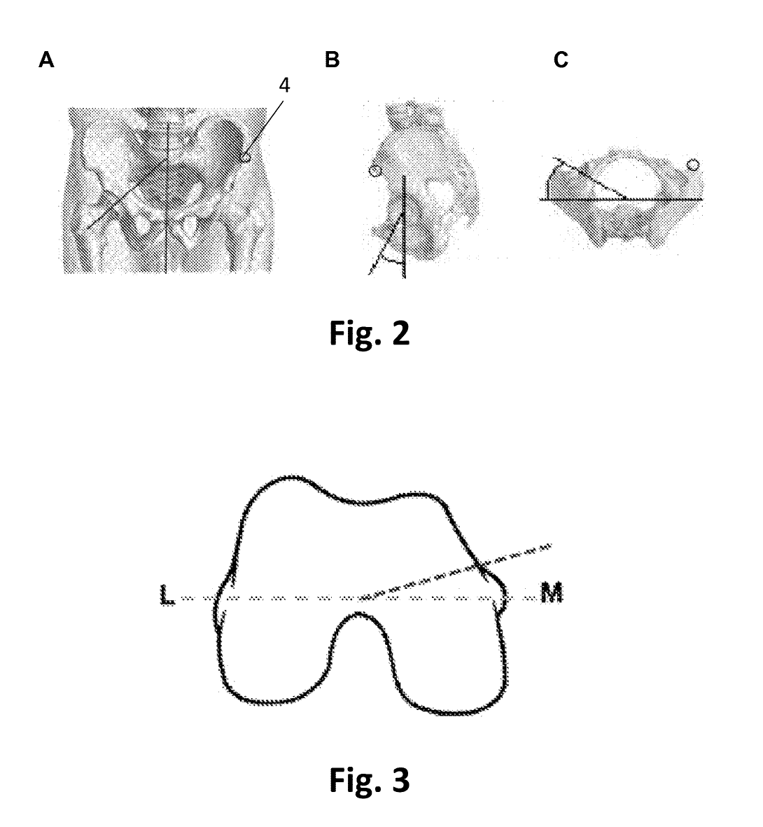

The angle of inclination is the angle between the axis of the acetabulum or acetabular implant and the sagittal plane, as projected onto the coronal plane (see FIG. 2A). The angle of forward flexion is the angle between the axis of the acetabulum or acetabular implant and the coronal plane, as projected onto the sagittal plane (see FIG. 2B). The angle of anteversion is the angle between the axis of the acetabulum or acetabular implant and the coronal plane, as projected onto the transverse plane (see FIG. 2C).

Looking at FIG. 3, the dashed line between L and M is the epicondylar axis. The acute angle defined by the two dashed lines is the angle of anteversion of the femur. The angle of anteversion in femur is the angle between the axis of the femoral neck and the epicondylar axis (of the distal femur).

The method and system provide precise information about the angles of inclination and forward flexion of the native bony acetabulum and prosthesis for proper implantation. These measurements and calculations are made in true relationship to the patient's pelvis and body axis during the time when the surgeon is preparing the host bone and handling the prosthesis and is inserting it into the host's native bone structure. The patient's pelvic axis is properly and accurately reproduced by connecting two identical spots on the pelvis, each on either side of the sagittal plane or midline. The anterior superior iliac spine 4 (or “ASIS”) is the most prominent bony landmark on the anterior aspect of the pelvis, readily identified with gentle palpation on all patients, regardless of their size, sex, or age. See FIG. 2a-c.

The measuring sensor units 2 are inexpensive, highly accurate, digital components able to communicate with an application software program running on a computer processor, personal computer (PC), or hand-held electronic device (e.g., smartphone or electronic tablet), to accurately determine the pelvic tilt as well as position angles of the acetabular implant while being or when placed in patient's bony pelvis. The determination of these angles can also be seen and read by the surgeon via a portable digital visual display, thereby removing the need for a PC. In one embodiment, the measuring system continuously monitors the patient's pelvic position, and as a consequence of this capability, the surgeon can effectively ensure an accurate angular placement of the acetabular prosthesis within the patient's native bone. The result will be optimum functionality of the joint and patient's satisfaction following surgery, a successful operation.

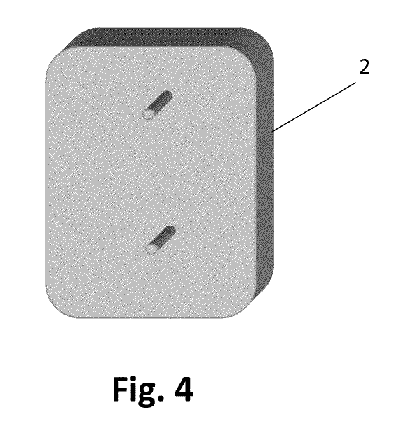

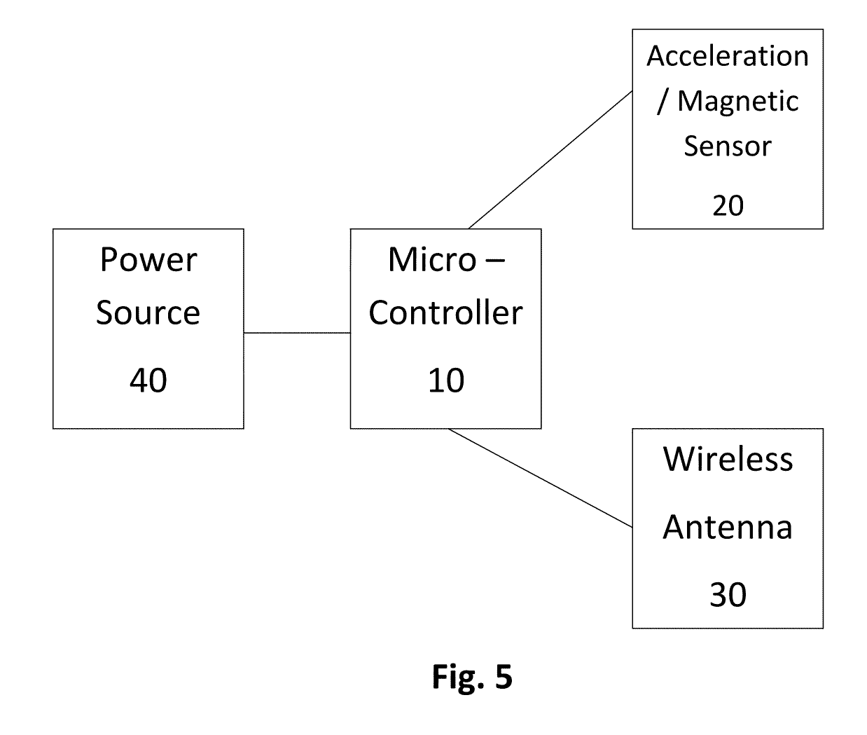

In particular, as seen in FIG. 4, an electronic position sensor 2 is used that is capable of sensing its orientation in 3-dimensional space and transmitting the information to the computer processor. It is attached to the patient's pelvis, optionally on the patient's ASIS, and transmits the position angles of the pelvis to a computer processor and application software. The position sensors 2 used herein have at least one orientation sensor 20 and at least one transmitter, or wireless antenna, 30. See FIG. 5. The transmitter 30 can be any of a variety of types used to transmit information, preferably wirelessly, to a computer or tablet. In one embodiment, the sensors 2 include a BLUETOOTH transceiver. The orientation sensors 20 preferably specify the tilt of the sensor with respect to orthogonal axes (such as x-y-z axes) and heading with respect to an external field. The external field measured by the orientation sensors 20 can be the Earth's magnetic field.

A sensor of the invention can include the following components:
a. A tilt sensor module and a direction sensor module 20 built in a MEMS (micro electro mechanical system) chip;
b. A Bluetooth module 30 for communication;
c. A micro controller 10 unit to operate the systems;
d. An internal power source 40; and
e. A printed circuit board onto which the other components can be placed.

In exemplary embodiments, the tilt sensor can be an accelerometer capable of measuring degrees of tilt from the true horizontal plane in three different axes. It can be used for sensing position and degree of the tilt of pelvis from vertical position. It can also be used for sensing the degree of tilt of the implant from horizontal plane.

The direction sensor can be a digital magnetometer capable of showing the direction of the axis of an object. The sensor can be used to sense the direction of the pelvis. An additional sensor can sense the implant vector of the acetabular cup when attached to an instrument that is used for placement of the cup.

One exemplary device that can be used is the LSM303DLHC system-in-package, available from STMicroelectronics, which features a 3D digital linear acceleration sensor and a 3D digital magnetic sensor. The output from such a system can be converted in software, firmware, or the like into the tilt data required by the invention.

The Bluetooth module 30 is used to provide a wireless mode of communication between the sensor and the central processor unit (such as a PC, tablet, etc.) running the application software. It wirelessly transfers the raw data from the sensor to the CPU where the application software receives the data and calculates the important position angles of the pelvis and implant. A graphical user interface can be provided to show the data to a user. The SPBT2632C2A, available from STMicroelectronics, is a small-sized Bluetooth module that provides sufficient functionality for the intended use of the sensor.

A microcontroller unit 10, such as the STM32F103RET6 ARM microcontroller available from STMicroelectronics, manages all of functions and performance of the main electronic components of the sensor.

The power source 40 can be three volt lithium CR1/3N batteries, for example, which can be used to power the sensor. Given the demands of sensing and transmitting of data in order to provide a surgeon with real time information, for example, on the tilt of a patient's pelvis, these batteries, which are sometimes used in products such as cameras and toys, are preferred over lower capacity batteries. In addition, in order to achieve battery life that is sufficient for the sensor to be used throughout a surgery, the firmware of the Bluetooth module can be adjusted to optimize its energy use and an oscillator can be added to aid in controlling power consumption.

An exemplary layout for the sensor of the invention is illustrated in FIG. 5. While certain specific electronic components have been mentioned herein and are disclosed in the circuit layout, it should be understood that a person of ordinary skill in the art may be able to select other comparable components within the spirit of the present invention.

An exemplary package for the sensor is illustrated in FIG. 4. A printed circuit board for providing the system of FIG. 5 can generally be flat, and providing a generally flat, rectangular package for the sensor can be convenient, and it can also provide visual cues to the surgeon when mounted on the patient in that the lines of the sensor can line up with the coronal and sagittal planes of the patient.

The tilt angles reported by a sensor mounted with respect to a patient's pelvic axis can be reported on a graphical user interface. That interface can report on the AP and or axial tilt of the pelvis. It can also provide a graphic showing the orientation of the patient's pelvis. If the surgeon wishes to have the patient's pelvis oriented differently for surgery, changes to the patient's position may be made with real time monitoring of the orientation of the pelvis.

Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims or those added to non-provisional applications claiming priority hereto.

What is claimed is:

1. A sensor for providing orientation data regarding a patient's pelvic axis, the sensor comprising:
a three axis tilt sensor;
a wireless communication module;
a power source; and
a microcontroller in electronic communication with the three axis tilt sensor, the wireless communication module, and the power source so as to control the three axis tilt sensor and the wireless communication module such that the sensor is configured to measure and report on an orientation of a pelvic axis of a patient to whom the sensor has been connected in a known orientation.

2. The sensor of claim 1, wherein the microcontroller further controls the three axis tilt sensor and the wireless communication module so that the battery can power the three axis tilt sensor, the wireless communication module, and the microcontroller for a period of at least one hour.

3. The sensor of claim 1, wherein the microcontroller further controls the three axis tilt sensor and the wireless communication module so that the battery can power the three axis tilt sensor, the wireless communication module, and the microcontroller for a period of at least four hours.

4. The sensor of claim 1, wherein the three axis tilt sensor includes a microelectromechanical accelerometer and a magnetometer.

5. The sensor of claim 1, further comprising a computer processor in wireless communication with the sensor, the computer processor including application software and a graphical user interface for converting data from the sensor into orientation information for the patient's pelvic axis and displaying the orientation information to a user.

6. A method for sensing and reporting on the orientation of a patient's pelvic axis, comprising:
providing sensor having:
a three axis tilt sensor;
a wireless communication module;
a power source; and
a microcontroller in electronic communication with the three axis tilt sensor, the wireless communication module, and the power source;
mounting the sensor on the patient in a known orientation with respect to the patient's pelvic axis;
sensing orientation data regarding the pelvic axis using the three axis tilt sensor;
communicating the orientation data using the wireless communication module.

7. The method of claim 6, wherein the microcontroller controls the three axis tilt sensor and the wireless communication module so that the battery can power the three axis tilt sensor, the wireless communication module, and the microcontroller for a period of at least one hour.

8. The method of claim 6, wherein the microcontroller controls the three axis tilt sensor and the wireless communication module so that the battery can power the three axis tilt sensor, the wireless communication module, and the microcontroller for a period of at least four hours.

9. The method of claim 6, wherein the three axis tilt sensor includes a microelectromechanical accelerometer and a magnetometer.

10. The method of claim 6, further comprising receiving of the wireless transmission of orientation data by a computer processor having an application program and a graphical user interface.

11. The method of claim 10, further comprising processing the orientation data by the application program and displaying orientation information regarding the patient's pelvic axis using the graphical user interface.
